(19) United States
(12) Patent Application Publication
(10) Pub. No.: US 2014/0328973 A1
(43) Pub. Date: Nov. 6, 2014

(54) METHOD OF RELEASING NICOTINE FROM CHEWING GUM

(71) Applicant: Bruno Provstgaard Nielsen, Vejle (DK)

(72) Inventor: Bruno Provstgaard Nielsen, Vejle (DK)

(21) Appl. No.: 14/367,679

(22) PCT Filed: Dec. 22, 2011
(86) PCT No.: PCT/DK2011/000157

§ 371 Date: Jun. 20, 2014

(51) Int. Cl. A61K 9/68; A23G 4/20; A61K 31/465; A23G 4/08

(52) U.S. Cl. CPC A61K 9/0058; A23G 4/08; A23G 4/20; A61K 31/465
USPC 426/2; 426/5

(57) ABSTRACT

A method of releasing nicotine from a compressed chewing gum tablet, wherein the chewing gum tablet includes two modules; a first module including nicotine and tablet material, and a second module including gum base and nicotine.

### FIELD OF THE INVENTION

The invention relates to a method of releasing nicotine from a compressed chewing gum tablet and a corresponding product.

### BACKGROUND OF THE INVENTION

Chewing gum has been used as a carrier of nicotine for decades. The reason for using chewing gum as a carrier is that chewing has advantageous properties in relation to release of nicotine, if the nicotine is mixed with the chewing gum. Several documents disclose different ways of modifying the nicotine in the chewing gum to ensure both a gradual steady release combined with an efficient transfer of nicotine through the mucous membrane of the mouth. The nicotine is thus delivered to the bloodstream via absorption in the mouth.

A common disadvantage related to all the above-mentioned disclosures is that users of nicotine-holding chewing gum uses the chewing gum as an alternative to smoking and that such users tend to prefer to smoke instead of the healthier alternative of nicotine gum if the user does not get the intended benefit instantaneous.

### SUMMARY OF THE INVENTION

The nicotine release obtainable according to the provisions of the invention is very suitable for counteracting craving and a user is therefore more attracted to obtaining the desired dose of nicotine through the nicotine chewing gum instead of getting the dose by means of smoking.

The invention relates to a method of releasing nicotine from a compressed chewing gum tablet,

the chewing gum tablet comprising two modules;

a first module comprising nicotine and tablet material,

a second module comprising gum base and nicotine and

wherein a user of said chewing gum tablet performs the steps of:
(a) positioning the chewing gum tablet in the mouth,
(b) dissolving the first module of the chewing gum tablet in a first period of time and thereafter,
(c) chewing the remaining part of the chewing gum tablet in a second period of time.

According to the invention, nicotine is transported from the chewing gum in two phases; an initial first phase where the absorption of nicotine in the mouth of the user is very effective and provides quick release without wasting too much of the released nicotine and a second phase where the nicotine is provided by nicotine released by the chewing gum, when it is chewed by the user.

The dissolving of the first module during the first period of time may be complete or incomplete. The important thing is that the user uses the first period of time to dissolve a part of or the complete first module to obtain an attractive and quick absorption of nicotine during the first period of time.

According to the invention, the initial release of nicotine is fast, but it also has a delaying factor, the dissolving, which ensures that the nicotine is not released from the chewing gum too fast, thereby effectively by-passing the mouth.

The dissolving may be more or less promoted by physical action of the user. The user may thus e.g. keep the tablet in the mouth, if the first layer e.g. is a melting tablet, the user may e.g. suck the tablet in the first period of time, the user may lick the tablet in the first period of time, etc.

A very attractive benefit obtained through the administering as set out according to the provisions of the invention, is that the first period of time is now availing both a fast release and an advantageous absorption of nicotine, which is superior to the absorption obtained, when dissolvable larger parts of chewing gums are provided and used conventionally.

According to the provisions of the invention, dissolving should be performed without chewing during the first period of time.

It is important according to the provisions that dissolving during the first period of time takes place in the mouth. The first module of the chewing gum tablet may, if being non-attached to the second module also be referred to as a orodispersibel tablet or tablet part.

In an embodiment of the invention, a user of said chewing gum tablet performs the steps of:
(a) positioning the chewing gum tablet in the mouth,
(b) dissolving the first module of the chewing gum tablet in a first period of time thereby releasing nicotine comprised in the first module,
(c) chewing the remaining part of the chewing gum tablet in a second period of time thereby releasing nicotine comprised in the second module.

According to an advantageous embodiment of the invention, the first period of time is primarily directed to release of nicotine comprised in the easy dissolvable part of the chewing gum tablet and the second period of time is primarily directed to release of nicotine comprised in the part of the chewing gum which is hardly dissolvable in the mouth due to the presence of the gum base contained in the module.

It is evident that some “cross-release” may occur within the scope of the invention, i.e. that a small amount of nicotine contained in the second module may be released during the first period of time and that a part of the nicotine contained in the first module of the chewing gum may be absorbed during the second period of time if there is still some part left of the first module when the user starts chewing on the chewing gum tablet. Still, if this happens, the benefit of applying the first module as the main source for nicotine absorption during the first period of time and the second module as the main source for nicotine absorption during the second period of time should be maintained by setting of appropriate first and second time periods. The same aspect should also be considered if a user for some reason chews during the first period of time. The main source of nicotine absorbed in the mouth during the first period of time should still be the nicotine contained in the dissolvable part of the tablet, the first module.

In an embodiment of the invention, a user of said chewing gum tablet performs the steps of:
(a) positioning the chewing gum tablet in the mouth,
(b) dissolving the first module of the chewing gum tablet in a first period of time,
(c) chewing the remaining part of the chewing gum tablet in a second period of time,
wherein the steps (a) to (c) are defined in instructions provided to the user together with said chewing gum tablet.

According to an advantageous embodiment, the user may be directed in the use of a chewing gum tablet according to instruction given together with the chewing gum tablet.

A benefit of this way of communicating the first period of time and optionally the second period of time is that the first period of time may be defined from the producer to fit the specific chewing gum tablet. In other words, the producer may design the first module a little more freely and dedicated to a specific task, as the first period of time may vary to fit different types of the first module.

The instructions may be communicated to the user either directly by means of instruction contained in or comprised in the package holding the chewing gum tablets. Appropriate means may include inlay instructions, printing in or outside the package. Alternatively, the instructions may be given by a mere reference at or in the package holding the chewing gum tablets to a specific information source, e.g. a web-site.

In an embodiment of the invention, the first period of time is the time it takes a user to dissolve the first module of the chewing gum tablet in the mouth.

The application of a first period of time where a user has to dissolve the first module of the chewing gum tablet renders the use of the chewing gum very intuitive, as the user under such circumstances simply has to dissolve the first module of the chewing gum as long as there is substantial amounts of the first module left. Thereafter, the user may feel in the mouth that the first module has been dissolved or almost dissolved and intuitively be incited to chew on the remaining part of the chewing gum and also refrain from chewing as long as there is something left of the first module.

In an embodiment of the invention, the first period of time has a duration of at least 2 seconds, preferably at least 5 seconds, more preferably at least 10 seconds.

In an embodiment of the invention, the first period of time has a duration of 10 to 240 seconds.

In an embodiment of the invention, the first period of time has a duration of 15 to 180 seconds.

In an embodiment of the invention, the first period of time has a duration of 20 to 40 seconds.

According to an advantageous embodiment of the invention, the first period time, where a user dissolves the dissolvable parts of the chewing gum in the mouth, should be longer than the time it e.g. takes to chew and swallow the main part of a coating of a conventional chewing gum or chew and swallow the tablet layer of a multilayered tablet in order to delay the initial dissolving phase and the resulting better uptake of nicotine in the mouth, but on the other hand also to ensure that the initial urgent need for counteracting craving is obtained.

In an embodiment of the invention, the first period of time is determined by the time, the user takes to dissolve all or a part of the first module when the user keeps the chewing gum tablet in the mouth.

According to an advantageous embodiment of the invention, the user will be given functional or relative instructions rather than absolute instructions defined by seconds or minutes. In such an embodiment, the user will be instruction to e.g. dissolve the tablet in the mouth until something physical and to the user perceivable occurs and then start chewing the remaining part of the tablet. Examples of such use could e.g. include that the user sucks, licks or simply keeps the chewing gum tablet in the mouth until e.g. the first module has been dissolved or substantially dissolved or e.g. until a change of taste occurs. The first perceivable reference point, i.e. initiating the chewing when a significant part of the first module is dissolved is straightforward. The other non-limiting example of a taste change my e.g. be obtained by having a further module integrated within the first module and wherein the further module comprises a flavour different from flavour included in the first module. Other measures may be applied in or on the chewing gum tablet to guide the user in relative terms rather than absolute definitions of the first time period.

In an embodiment of the invention, the second period of time has a duration of at least 10 seconds, preferably at least 60 seconds, more preferably at least 4 minutes.

In an embodiment of the invention, the second period of time has a duration of 5 to 30 minutes.

According to an advantageous embodiment of the invention, the second period of time should be sufficient to keep the absorption of nicotine from the chewing gum tablet, of primarily from the second module, high enough during a relatively long time interval. For this purpose, such steady and relatively long release and absorption though the mouth may be obtained by means of known nicotine chewing gum formulation.

In an embodiment of the invention, the first period of time is shorter than the second period of time.

In an embodiment of the invention, said second module comprising gum base and nicotine is formed by means of compressed granules, at least a part of the compressed granules comprising gum base.

In an embodiment of the invention, the compressed granules comprising gum base further comprises chewing gum ingredients.

In an embodiment of the invention, at least a part of the nicotine contained in the second module is mixed into the compressed granules comprising gum base prior to granulation.

In an embodiment of the invention, at least a part of the nicotine contained in the second module is contained in compressed granules having no gum base.

In an embodiment of the invention, the nicotine in the second module is buffered.

In an embodiment of the invention, the compressed chewing gum tablet comprises buffer in an amount of 0.01-50%.

In an embodiment of the invention, a part of the second modules is comprised by compressed granules comprising one or more chewing gum ingredients selected from the group consisting of bulk sweeteners, flavors, dry binders, tabletting aids, anti-caking agents, emulsifiers, antioxidants, or any combination thereof.

In an embodiment of the invention, said gum base comprises two or more ingredients selected from the group consisting of elastomers, elastomer plasticizers, resins, polyvinyl acetate, hydrogenated resins, polyterpene resins, fillers, hydrogenated starch hydrolysate, fats and waxes, or any combination thereof.

In an embodiment of the invention, said chewing gum ingredients are selected from the group consisting of bulk sweeteners, flavors, dry binders, tabletting aids, anti-caking agents, emulsifiers, antioxidants, high intensity sweeteners, colors, or any combination thereof.

In an embodiment of the invention, the bulk sweetener is selected from the group of direct-compressible sweeteners, such as sorbitol.

In an embodiment of the invention, the first module comprising nicotine and tablet materials is formed of compressed granules of tablet material and granules comprising nicotine.

In an embodiment of the invention, at least a part of the granules of the first module further comprises chewing gum ingredients.

In an embodiment of the invention, at least a part of the granules of the first module further comprises chewing gum ingredients selected from the group consisting of bulk sweeteners, flavors, dry binders, tabletting aids, anti-caking agents, emulsifiers, antioxidants, high intensity sweeteners, colors, or any combination thereof.

In an embodiment of the invention, the bulk sweetener of the first module has a low compressibility.

In an embodiment of the invention, at least a part of the bulk sweetener of the first module is selected from the group of compressible sweeteners having a compressibility which is lower than the compressibility of the compressible bulk sweeteners of the second module.

According to a preferred embodiment of the invention, the first module should be dissolvable in the mouth of a user without chewing the module.

In an embodiment of the invention, the first module is accessible for saliva when positioned in the mouth.

In an embodiment of the invention, the first module is a fast disintegrating module and comprises components typically applied in fast disintegrating tablets (FDT).

In an embodiment of the invention, the first module is comprised of compressed particles of nicotine and tablet material, wherein the tablet material comprises mucoadhesives and/or disintegrants.

In an embodiment of the invention, the first module should be designed to be dissolved/disintegrated faster than normal tables modules of compressed chewing gum. Specific measures, such as disintegrants known in the art may be applied. Other measure such as choosing the applied bulk-sweeteners in the first module to fit the purpose may also be attractive. Numerous different bulk sweeteners may be applied for the purpose.

In an embodiment of the invention, the first module is gum-base free.

According to a preferred embodiment of the invention, the first module should be essentially dissolvable in the mouth and this dissolvability may be obtained by the application of no or little gum base in the dissolvable portion.

In an embodiment of the invention, the content of gum base in the first module is less than 10% by weight of the first module.

In an embodiment of the invention, the nicotine in the first module is buffered.

In an embodiment of the invention, the nicotine in the first module is non-buffered.

In an embodiment of the invention, the nicotine in the first module is non-buffered and wherein the nicotine in the second module is buffered.

In an embodiment of the invention, the nicotine types in the first and second modules are different.

In an embodiment of the invention, the nicotine contained in the chewing gum tablet comprises nicotine added as a salt such as nicotine bitartrate, nicotine pftalate, nicotine polacrilex, nicotine sulphate, nicotine tartrate, nicotine citrate, or nicotine lactate.

In an embodiment of the invention, the nicotine contained in the first module comprises nicotine salts.

In an embodiment of the invention, the nicotine contained in the second module comprises nicotine bound to an ion exchange resin.

In an embodiment of the invention, the nicotine contained in the first module comprises nicotine salts and wherein the nicotine contained in the second module comprises nicotine bound to an ion exchange resin.

In an embodiment of the invention, the nicotine contained in the first module comprises nicotine salts and wherein the nicotine contained in the second module comprises nicotine bound to an ion exchange resin and wherein the nicotine comprised in the second module is buffered.

In an embodiment of the invention, the nicotine contained in the second module comprises nicotine bound to an inorganic filler.

In an embodiment of the invention, the first and second module both comprises flavour and sweetener.

According to a very preferred embodiment of the invention, both modules should contain sweetener and flavour to give a user a pleasant experience during both first and second time period of time.

In an embodiment of the invention, the sweeteners applied in the first and second modules differs in compressibility and where the compressibility of the sweetener in the first module is lower than the compressibility of the sweetener in the second module.

In an embodiment of the invention, the chewing gum tablet modules are in the form of layers.

According to various embodiments, the modules may be shaped in different ways. However, according to the present invention the term “module” should be understood to be an independent recognizable part, i.e. not just e.g. a single particle in a powder mixture. Consequently, a module indicates an element with a volume of at least 3 mm³, preferably more.

In an embodiment of the invention, the chewing gum has one or further additional modules.

In an embodiment of the invention, the second module is encapsulated in the first module.

In an embodiment of the invention, the second module is non-encapsulated.

According to a preferred embodiment of the invention, the first module should be directly accessible to the saliva in the mouth, thereby ensuring that the initial dissolving may be performed without initial chewing.

In an embodiment of the invention, said first and second modules of said compressed chewing gum tablet forms at least two individual coherent compressed modules.

In an embodiment of the invention, the first and second modules are compressed in two compressing steps.

A way of gathering the two modules may be to initially compress the tablet module, the first module, and then compress the gum base containing module, the second module, on top of the other, thereby also gathering the two modules.

In an embodiment of the invention, the first and second modules are compressed in one compressing step.

Another way of gathering the two modules may simply be to compress the first module and the second module in one compression step, thereby obtaining the compression of the modules and the gathering in the same step.

In an embodiment of the invention, said compressed chewing gum tablet comprises at least one further module.

The chewing gum tablet may in principle comprise one or several further modules in addition to the first and second module as long as the first and second module are arranged mechanically to serve the purpose of facilitation of a first dissolving phase resulting in absorption of nicotine in the mouth of the user from the first module in a first period of time and a second chew phase resulting in absorption of nicotine in the mouth of a user from the second module in a second period of time.

Moreover, the invention relates to a chewing gum collection comprising at least two chewing gum tablets, the chewing gum tablets comprising two modules;

a first module comprising nicotine and tablet material,

a second module comprising gum base and nicotine,

the chewing gum collection further comprising instructions to a user of the chewing gum tablets,

the instructions directing a user of the chewing gum tablets to performs the steps of:
(a) positioning the chewing gum tablet in the mouth,
(b) dissolving the first module of the chewing gum tablet in a first period of time thereby releasing nicotine comprised in the first module and thereafter
(c) chewing the remaining part of the chewing gum tablet in a second period of time thereby releasing nicotine comprised in the second module.

In an embodiment of the invention, the chewing gum collection comprises the chewing gum tablets contained in a packaging.

In an embodiment of the invention, the chewing gum collection comprises the chewing gum tablets contained in a packaging comprising a blister packaging, a plastic container, a bag or a box.

In an embodiment of the invention, the instructions are contained in or attached to the packaging.

In an embodiment of the invention, information attached to or contained in the packaging directs the user to a source of information where the instructions related to the chewing gum collection contained in the packaging.

Moreover, the invention relates to a chewing gum collection, wherein the instructions refer to any of the provisions defined herein.

In an embodiment of the invention, the chewing gum tablet is a chewing gum tablet as defined herein.

### DETAILED DESCRIPTION

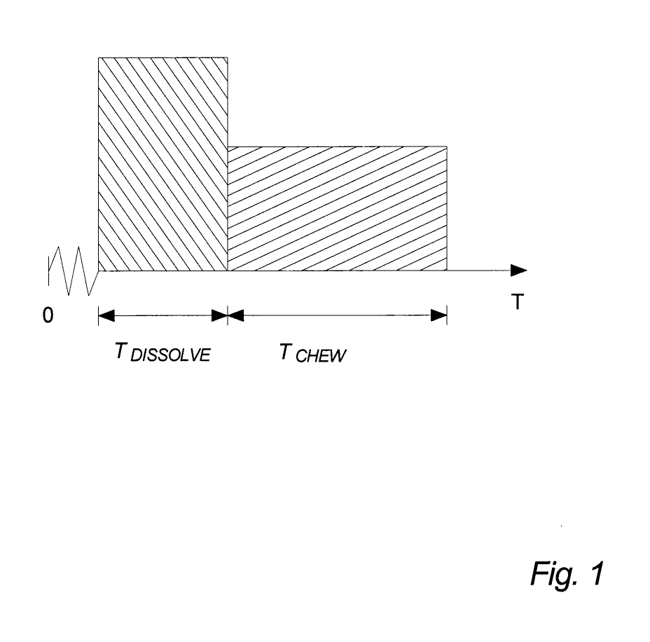

FIG. 1 illustrates some basic understanding, which may serve to explain some relevant features of the present invention.

The FIGURE shows an axis T, referring to time.

The time T progresses from t=0 as illustrated to the left of the FIGURE and is determined by the time a user puts a chewing gum tablet as explained in the present application into the mouth.

In a first period of time, T_{DISSOLVE}, the user dissolves the dissolvable part of the chewing gum tablet, preferably without chewing the tablet.

In principle, the time scale illustrates that the starting time of the dissolving period may be later than t=0 where a user puts the chewing gum tablet into the mouth. In practice t=0, i.e. the time where a user puts the chewing gum into the mouth, will typically be the starting of the dissolving of the chewing gum tablet in the mouth of the user and thereby the starting of the first period of time. The starting time may deviate from t=0 as long as the dissolving phase, the first period of time, results in a substantial release of nicotine from the chewing gum tablet.

In a second period of time, T_{CHEW}, the user chews the remaining part of the chewing gum tablet. Optional remains from the dissolvable part of the chewing gum tablet will then be chewed together with the non-dissolvable part of the chewing gum.

The amount of released nicotine during both phases may among many factors depend on the amount and type of nicotine included in the first and second modules.

The release and absorption of nicotine from the second module will largely be established as in prior art nicotine chewing gum, where the release of nicotine is obtained according to certain established release-profiles and by means of suitable chewing gum structures and formulations. Moreover such release may also be adjusted e.g. by means of coating of the particles to be released.

The amount of released and absorbed nicotine from the first module is more critical and may depend strongly on whether the user is instructed to use the chewing gum properly, i.e. that the user should avoid chewing the chewing gum during the first period of time and then start chewing after a certain period of time. If a user starts chewing too soon, a substantial portion of the nicotine from the first module may be swallowed because of poor absorption in the mouth. Since nicotine, when swallowed, can cause adverse gastrointestinal symptoms, such as hiccupping and nausea, a user should be well instructed in the use of the chewing gum. Another side-effect of such wrong use of the chewing gum tablet is that expensive nicotine is simply lost as the digested portion of the nicotine gives little of the desired effect.

It should be noted that the Y-axis is undefined. That is because the Y-axis only serves to illustrate that an undisclosed amount of nicotine is released from the chewing gum tablet during the two periods of time. The FIGURE does not give any essential information regarding the effective amount over time or in total. This will depend of content of nicotine in the modules/layers of the chewing gum tablet, gum base structure, etc.

The below examples are specific embodiments of the invention.

### EXAMPLES

### Example 1

### Preparation of Gum Base and Preparation of Granules

Gum base is prepared with a content of absorption enhancer and gum base is prepared without a content of absorption enhancer. There are applied enhancers such as pH control agents as shown in the compositions outlined in table 1.

TABLE 1
Gum base compositions. Amounts are given
in percent by weight of each composition.

| Component | GB 1 | GB 2 |
|---|---|---|
| Elastomer | 10 | 10 |
| PVA | 21 | 21 |
| Natural resin | 29 | 29 |
| Filler | 11.5 | 16.5 |
| Na₂CO₃-pH control agent | 5 | 0 |
| Emulsifier | 4 | 4 |
| Softener (wax and fat) | 19.4 | 19.4 |
| Antioxidant | 0.1 | 0.1 |
| | (900 ppm) | (900 ppm) |

The preparation of the gum base is carried out by first adding elastomer, polyvinyl acetate, filler and sodium carbonate to a heated (about 120° C.) and running z-blade mixer. After about twenty minutes of mixing, natural resin is added to the running mixer and mixing is continued for about five minutes followed by addition of further natural resin. After about five minutes of continued mixing, emulsifier and further elastomer are added to the running mixer, and mixing is continued for about five minutes before addition of softener and antioxidant to the running mixer. Mixing is continued for about half an hour to one hour, and the final gum base mass is emptied from the mixer into coated or lined pans, extruded or cast into any desirable shape. Those skilled in the art will recognize that many variations of the above-described procedure may be followed.

To form the gum base as granules, the prepared gum base is transferred either directly or in the form of pellets to an extruder (here a Leistritz ZSE/BL 360 kw 104, available from Leistritz GmbH, Germany), which extrudes the gum base through a die plate into a liquid filled chamber (here a granulator A5 PAC 6, available from GALA GmbH, Germany). Descriptions of the extruder and the granulator may be found in e.g. WO 2004/098305, incorporated herein by reference.

The already prepared gum base composition is added at a first inlet of the extruder. Menthol flavor crystals in an amount of about 3% by weight of the gum base is dosed to a second inlet and mixed into the gum base composition in the extruder. The addition of menthol could be omitted to form gum base granules entirely based on the already prepared gum base. However, an improved taste and texture of the granules can be obtained by adding additional ingredients such as the above or other flavors or additives.

The extruder delivers the gum base-comprising composition at a feed rate of 400 kg/h to the die plate. An extruder screw speed set at 247 rpm is applied, and the temperature in the extruder is in the range of 40° C. to 70° C. along about ¾ of the extruder barrel length, until the composition passes a heating device in the outlet end of the extruder. Here the composition is heated to an extruder exit temperature of about 109° C. The extruder and the granulator produce a pressure difference of about 70-75 bar.

The composition is extruded through the die plate, which is here a die plate having 696 holes with a diameter of 0.36 mm and being heated to a temperature of about 177° C. In the granulator chamber the extruded composition is cut to granules by a cutter with 8 blades and cutter speed set at 1999 rpm. The particles are cooled and transported to the strainer unit (here a centrifugal dryer TWS 20, available from GALA GmbH, Germany) in water with temperature about 11° C. and flow about 22 m³/h. The average cooling and transport time in water is approx. 60 seconds. The particle rate is 400 kg/h and the average diameter of the obtained particles is here obtained to be 0.93 mm.

The cooling and transport stage carried out in water in this example could be carried out in other media such as e.g. air as well. Also various alternative apparatuses, die plates, settings, etc. could be applied in order to obtain smaller or larger average particle sizes of the prepared granules.

The granules are applied in the compressed chewing gum according to the below examples.

### Example 2

### Two-Layer Compressed Gum Tablets

Two-layer tablets with contents of the pharmaceutically active ingredients of Nicotine are prepared with the compositions given in table 2.

TABLE 2
Chewing gum compositions of two-layer chewing gum tablets
comprising pharmaceutically active sources of Nicotine.
The Nicotine strength is 2 mg. Amounts are given in
percent by weight of each composition.

| | Composition no. | | | |
|---|---|---|---|---|
| Components | A | B | C | D |
| Layer 1, weight [mg] | 1000 | 1000 | 1000 | 1000 |
| GB 1, granules of ex. 1 | 50 | 0 | 50 | 0 |
| GB 2, granules of ex. 1 | 0 | 50 | 0 | 50 |
| Bulk sweetener | 23.12 | 15.12 | 23.12 | 15.12 |
| Effersoda (encapsulated | 0 | 8 | 0 | 8 |
| buffer system) | | | | |
| Peppermint powder | 2 | 2 | 2 | 2 |
| Menthol powder | 1 | 1 | 1 | 1 |
| Sucralose | 0.2 | 0.2 | 0.2 | 0.2 |
| Acesulfame potassium | 0.1 | 0.1 | 0.1 | 0.1 |
| Tabletting aid | 0.5 | 0.5 | 0.5 | 0.5 |
| Layer 2, weight [mg] | 300 | 300 | 300 | 300 |
| Isomalt GalenIQ720 | 20.11 | 20.11 | 20.71 | 20.71 |
| Peppermint powder | 1 | 1 | 1 | 1 |
| Menthol powder | 0.25 | 0.25 | 0.25 | 0.25 |
| NPR with nicotine load | 1.12 | 1.12 | 0 | 0 |
| 15.3% | | | | |
| Nicotine Bitartrate | 0 | 0 | 0.52 | 0.52 |
| Na₂CO₃ powder | 0.5 | 0.5 | 0.5 | 0.5 |
| Acesulfame potassium | 0.1 | 0.1 | 0.1 | 0.1 |

The compositions of layer 1 are obtained by blending the gum base granules, bulk sweetener, buffer, high potency sweeteners, flavors and tabletting aid and for each composition. The compositions of layer 2 are mixed according to table 2. The chewing gum tablets are compressed in a tabletting press, where tablet weights are adjusted to 1300 g. The bulk sweetener choosen for layer 2 in the tablets A to D facilitates that the surface of layer 2 is hard and smooth. Other bulk sweeteners like Isomalt GalenIQ720, Mannitol, Sorbitol, and Xylitab could have been choosen. The selection is often based on the stability requirement and compability to other ingredients in the tablet.

Moreover, the tablets may by applied according to the provisions of the present invention by a user who during a first period of time dissolves Layer 2 in the mouth, thereby releasing nicotine contained in the layer 2 relatively fast, but still slow enough to ensure that a relatively large amount of the nicotine contained in layer 2 as absorbed in the mouth of the user instead being swallowed, which would largely be the case if the chewing gum was simply chewed as from the beginning.

The user may advantageously be instructed to dissolve the tablet in the mouth until all or most of layer 2 has been dissolved. The user may thus simply feel that the dissolvable module, layer 2, is gone or almost gone and then start chewing the remaining of a layer 1 and optionally layer 2, thereby gradually releasing the nicotine in the remaining tablet, primarily layer 1.

### Example 3

### Two-Layer-Gum Tablets

Four alternative two-layer tablets are described below. The below tablets E, F, G and H are very advantageous for use according to the provisions of the invention, i.e. as a nicotine releasing chewing gum tablet, where a user dissolves the tablet in a first time period and subsequently chews the remaining part of the tablet.

The two-layer gum tablets are prepared with the compositions given in table 3.

TABLE 3
Chewing gum compositions of chewing gum tablets comprising
pharmaceutically active sources of Nicotine. Amounts are
given in percent by weight of each composition. The Nicotine
strength is 1.5 mg in the layer 1 and 0.5 mg in layer 2,
but other combinations could be applied.

| | Composition no. | | | |
|---|---|---|---|---|
| Components | E | F | G | H |
| Layer 1, weight [mg] | 1000 | 1000 | 1000 | 1000 |
| GB 1, granules of ex. 1 | 50 | 0 | 50 | 0 |
| GB 2, granules of ex. 1 | 0 | 50 | 0 | 50 |
| Bulk sweetener | 22.3 | 14.3 | 22.3 | 14.3 |
| NPR with nicotine load | 0.8 | 0.8 | 0.8 | 0.8 |
| 15.13% | | | | |
| Effersoda (encapsulated | 0 | 8 | 0 | 8 |
| buffer system) | | | | |
| Peppermint powder | 2 | 2 | 2 | 2 |
| Menthol powder | 1 | 1 | 1 | 1 |
| Sucralose | 0.2 | 0.2 | 0.2 | 0.2 |
| Acesulfame potassium | 0.1 | 0.1 | 0.1 | 0.1 |
| Tabletting aid | 0.5 | 0.5 | 0.5 | 0.5 |
| Layer 2, weight [mg] | 300 | 300 | 300 | 300 |
| Mannitol (Pearlitol | 21.13 | 21.13 | 0 | 0 |
| Flash) | | | | |
| Maltitol P200 Sweetpearl | 0 | 0 | 18.63 | 18.63 |
| Disintegrant | 0 | 0 | 2.5 | 2.5 |
| Peppermint powder | 1 | 1 | 1 | 1 |
| Menthol powder | 0.25 | 0.25 | 0.25 | 0.25 |
| Nicotine Bitartrate with | 0.12 | 0.12 | 0.12 | 0.12 |
| purity 99.7% | | | | |
| Na₂CO₃ powder | 0.5 | 0.5 | 0.5 | 0.5 |
| Acesulfame potassium | 0.1 | 0.1 | 0.1 | 0.1 |

The compositions of layer 1 are obtained by blending the gum base granules, NPR, bulk sweetener, buffer, high potency sweeteners, flavors and tabletting aid. The compositions of layer 2 are mixed and compressed in a tabletting press.

In the present example, Layer 2 may be compressed at a lower pressure when compared to conventional compressed gum.

The purpose of layer 2 in the chewing gum product is to dissolve quickly within 30 seconds and thereby release the Nicotine bitartrate and if applicable buffer. Furthermore, release of flavors and high potenty sweeteners for taste masking.

The acceptance criteria for friability should be fulfilled so packaging of the resulting chewing gum tablets is possible, but in this embodiment, the bulk sweetener should have relatively good compressibility and still have fast disintegration as compared to the previous example. A bulk sweetener like the above-applied Mannitol Pearlitol Flash from Roquette is suitable for this purpose. Another example is Pharmaburst 500 from SPI Pharma.

Another solution for making the chewing gum tablet is to provide the formulation with a disintegrant in layer 2 together with a bulk sweetener that is not ideal for fast disintegration by itself. This excipient will speed up the process of dissolving the layer 2 when the tablet is exposed to human saliva.

The tablets E to H may by applied according to the provisions of the present invention by a user wherein the user during a first period of time dissolves Layer 2 in the mouth, thereby releasing nicotine contained in the layer 2 relatively fast, but still slow enough to ensure that a relatively large amount of the nicotine contained in layer 2 is absorbed in the mouth of the user instead of being swallowed, which would largely be the case if the chewing gum was simply chewed as from the beginning.

The user may advantageously be instructed to dissolve the tablet in the mouth until all or most of layer 2 has been dissolved. The user may thus simply feel that the dissolvable module, layer 2, is gone or almost gone and then start chewing the remaining part of layer 1 and optionally layer 2, thereby gradually releasing the nicotine in the remaining tablet, primarily layer 1.

The two-layer chewing gum tablets E to H show even better qualities as regards control of Nicotine release and thereby Nicotine craving as compared with the two-layer compressed gum tablets described in example 2, as layer 2 has a primary content of bulk sweetener with a relatively low compressibility. According to advantageous embodiments of the invention, it is preferred to use bulk sweetener having a low compressibility in the dissolvable module, to ensure that the user may obtain a release from the module relatively fast when the chewing gum tablet has been put into the mouth.

As an alternative, or a supplement, to these choices of compressible sweeteners, the sweeteners in the dissolvable module may be compressed by a reduced pressure when compared with e.g. the pressure applied for the tablets A to D.

In some embodiments nicotine is added as a salt such as nicotine bitartrate, nicotine pftalate, nicotine polacrilex, nicotine sulphate, nicotine tartrate, nicotine citrate, or nicotine lactate.

The composition of gum base formulations can vary substantially depending on the particular product to be prepared and on the desired masticatory and other sensory characteristics of the final product. However, typical ranges of the above gum base components are: 5 to 80% by weight of elastomeric compounds, 5 to 80% by weight of elastomer plasticizers, 0 to 40% by weight of waxes, 5 to 35% by weight of softener, 0 to 50% by weight of filler, and 0 to 5% by weight of miscellaneous ingredients such as antioxidants, colorants, etc. The gum base may comprise about 5 to about 95% by weight of the chewing gum, more commonly; the gum base comprises 10 to about 60% by weight of the gum.

Elastomers provide the rubbery, cohesive nature to the gum, which varies depending on this ingredient's chemical structure and how it may be compounded with other ingredients. Elastomers suitable for use in the gum base and gum of the present invention may include natural or synthetic types.

Elastomer plasticizers vary the firmness of the gum base. Their specificity on elastomer inter-molecular chain breaking (plasticizing) along with their varying softening points cause varying degrees of finished gum firmness and compatibility when used in gum base. This may be important when one wants to provide more elastomeric chain exposure to the alkanic chains of the waxes.

If desired, conventional elastomers or resins may be supplemented or substituted by biodegradable polymers.

Agglomeration which may be used on e.g. tablet material and active ingredients in an embodiment of the invention may be performed for instance by fluid bed agglomeration, a process known to the person skilled in the art.

The chewing gum may include component known in the chewing gum art. For example, the chewing gum may include elastomers, bulking agents, waxes, elastomer solvents, emulsifiers, plasticizers, fillers, and mixtures thereof.

The chewing gum according to the invention may comprise coloring agents. According to an embodiment of the invention, the chewing gum may comprise color agents and whiteners such as FD&C-type dyes and lakes, fruit and vegetable extracts, titanium dioxide and combinations thereof.

Further useful chewing gum base components include antioxidants, e.g. butylated hydroxytoluene (BHT), butyl hydroxyanisol (BHA), propylgallate and tocopherols, and preservatives.

A gum base formulation may, in accordance with the present invention, comprise one or more softening agents e.g. sucrose esters including those disclosed in WO 00/25598, which is incorporated herein by reference, tallow, hydrogenated tallow, hydrogenated and partially hydrogenated vegetable oils, cocoa butter, degreased cocoa powder, glycerol monostearate, glyceryl triacetate, lecithin, mono-, di- and triglycerides, acetylated monoglycerides, lanolin, sodium stearate, potassium stearate, glyceryl lecithin, propylene glycol monostearate, glycerine, fatty acids (e.g. stearic, palmitic, oleic and linoleic acids) and combinations thereof. As used herein the term “softener” designates an ingredient, which softens the gum base or chewing gum formulation and encompasses waxes, fats, oils, emulsifiers, surfactants and solubilisers.

To soften the gum base further and to provide it with water-binding properties, which confer to the gum base a pleasant smooth surface and reduce its adhesive properties, one or more emulsifiers is/are usually added to the composition, typically in an amount of 0 to 18% by weight, preferably 0 to 12% by weight of the gum base. Useful emulsifiers can include, but are not limited to, glyceryl monostearate, propylene glycol monostearate, mono- and diglycerides of edible fatty acids, lactic acid esters and acetic acid esters of mono- and diglycerides of edible fatty acids, acetylated mono and diglycerides, sugar esters of edible fatty acids, Na-, K-, Mg- and Ca-stearates, lecithin, hydroxylated lecithin and the like and mixtures thereof are examples of conventionally used emulsifiers which can be added to the chewing gum base. In case of the presence of a biologically or pharmaceutically active ingredient as defined below, the formulation may comprise certain specific emulsifiers and/or solubilisers in order to disperse and release the active ingredient.

Waxes and fats are conventionally used for the adjustment of the texture and for softening of the chewing gum base when preparing chewing gum bases. In connection with the present invention, any conventionally used and suitable type of natural and synthetic wax and fat may be used, such as for instance rice bran wax, polyethylene wax, petroleum wax (refined paraffin and microcrystalline wax), sorbitan monostearate, tallow, propylene glycol, paraffin, beeswax, carnauba wax, candelilla wax, cocoa butter, degreased cocoa powder and any suitable oil or fat, as e.g. completely or partially hydrogenated vegetable oils or completely or partially hydrogenated animal fats.

A chewing gum base formulation may, if desired, include one or more fillers/texturisers including as examples, magnesium and calcium carbonate, sodium sulphate, ground limestone, silicate compounds such as magnesium and aluminum silicate, kaolin and clay, aluminum oxide, silicium oxide, talc, titanium oxide, mono-, di- and tri-calcium phosphates, cellulose polymers, such as wood, and combinations thereof.

In addition to a water insoluble gum base portion, a typical chewing gum includes a water soluble bulk portion and one or more flavoring agents. The water-soluble portion may include bulk sweeteners, high-intensity sweeteners, flavoring agents, softeners, emulsifiers, colors, acidulants, buffering agents, fillers, antioxidants, and other components that provide desired attributes.

Combinations of sugar and/or non-sugar sweeteners can be used in the chewing gum formulation processed in accordance with the invention. Additionally, the softener may also provide additional sweetness such as aqueous sugar or alditol solutions.

Useful sugar sweeteners are saccharide-containing components commonly known in the chewing gum art including, but not limited to, sucrose, dextrose, maltose, dextrins, trehalose, D-tagatose, dried invert sugar, fructose, levulose, galactose, corn syrup solids, and the like, alone or in combination.

Sorbitol can be used as a non-sugar sweetener. Other useful non-sugar sweeteners include, but are not limited to, other sugar alcohols such as mannitol, xylitol, hydrogenated starch hydrolysates, maltitol, isomaltol, erythritol, lactitol and the like, alone or in combination.

High-intensity artificial sweetening agents can also be used alone or in combination with the above sweeteners. Preferred high-intensity sweeteners include, but are not limited to sucralose, aspartame, salts of acesulfame, alitame, neotame, twinsweet, saccharin and its salts, cyclamic acid and its salts, glycyrrhizin, dihydrochalcones, thaumatin, monellin, stevioside and the like, alone or in combination. In order to provide longer lasting sweetness and flavor perception, it may be desirable to encapsulate or otherwise control the release of at least a portion of the artificial sweetener. Techniques such as wet granulation, wax granulation, spray drying, spray chilling, fluid bed coating, coascervation, encapsulation in yeast cells and fiber extrusion may be used to achieve the desired release characteristics. Encapsulation of sweetening agents can also be provided using another chewing gum component such as a resinous compound.

Usage level of the high-intensity artificial sweetener will vary considerably and will depend on factors such as potency of the sweetener, rate of release, desired sweetness of the product, level and type of flavor used and cost considerations. Thus, the active level of high-potency artificial sweetener may vary from about 0 to about 8% by weight, preferably 0.001 to about 5% by weight. When carriers used for encapsulation are included, the usage level of the encapsulated sweetener will be proportionately higher.

If a low-calorie gum is desired, a low-caloric bulking agent can be used. Examples of low caloric bulking agents include polydextrose, Raftilose, Raftilin, fructooligosaccharides (NutraFlora®), palatinose oligosaccharides; guar gum hydrolysates (e.g. Sun Fiber®) or indigestible dextrins (e.g. Fibersol®). However, other low-calorie bulking agents can be used.

The chewing gum according to the present invention may contain aroma agents and flavoring agents including natural and synthetic flavorings e.g. in the form of natural vegetable components, essential oils, essences, extracts, powders, including acids and other substances capable of affecting the taste profile. Examples of liquid and powdered flavorings include coconut, coffee, chocolate, vanilla, grape fruit, orange, lime, menthol, liquorice, caramel aroma, honey aroma, peanut, walnut, cashew, hazelnut, almonds, pineapple, strawberry, raspberry, tropical fruits, cherries, cinnamon, peppermint, wintergreen, spearmint, eucalyptus, and mint, fruit essence such as from apple, pear, peach, strawberry, apricot, raspberry, cherry, pineapple, and plum essence. The essential oils include peppermint, spearmint, menthol, eucalyptus, clove oil, bay oil, anise, thyme, cedar leaf oil, nutmeg, and oils of the fruits mentioned above.

The chewing gum flavor may be a natural flavoring agent, which is freeze-dried, preferably in the form of a powder, slices or pieces or combinations thereof. The particle size may be less than 3 mm, less than 2 mm or more preferred less than 1 mm, calculated as the longest dimension of the particle. The natural flavoring agent may be in a form where the particle size is from about 3 μm to 2 mm, such as from 4 μm to 1 mm. Preferred natural flavoring agents include seeds from fruit e.g. from strawberry, blackberry and raspberry.

Various synthetic flavors, such as mixed fruit flavors may also be used in the present chewing gum centers. As indicated above, the aroma agent may be used in quantities smaller than those conventionally used. The aroma agents and/or flavors may be used in the amount from 0.01 to about 30% by weight of the final product depending on the desired intensity of the aroma and/or flavor used. Preferably, the content of aroma/flavor is in the range of 0.2 to 5%, more preferably 0.5 to 3%, by weight of the total composition.

In an embodiment of the invention, the flavoring agents comprise natural and synthetic flavorings in the form of natural vegetable components, essential oils, essences, extracts, powders, including acids and other substances capable of affecting the taste profile.

In one embodiment of the invention, the flavor may be used as taste masking in chewing gum comprising active ingredients, which by themselves have undesired taste or which alter the taste of the formulation.

Active ingredients may advantageously be applied in a chewing gum according to am embodiment the invention. Active ingredients generally refer to those ingredients that are included in a delivery system and/or compressible chewing gum composition for the desired end benefit they provide to the user

1-59. (canceled)

60. Method of releasing nicotine from a compressed chewing gum tablet, the chewing gum tablet comprising two modules;
a first module comprising nicotine and tablet material,
a second module comprising gum base and nicotine, and
wherein a user of said chewing gum tablet performs the steps of:
(a) positioning the chewing gum tablet in the mouth,
(b) dissolving the first module of the chewing gum tablet in a first period of time, and thereafter
(c) chewing the remaining part of the chewing gum tablet in a second period of time.

61. Method of releasing nicotine according to claim 60, wherein a user of said chewing gum tablet performs the steps of:
(a) positioning the chewing gum tablet in the mouth,
(b) dissolving the first module of the chewing gum tablet in a first period of time,
(c) chewing the remaining part of the chewing gum tablet in a second period of time,
wherein the steps (a) to (c) are defined in instructions provided to the user together with said chewing gum tablet.

62. Method of releasing nicotine according to claim 60, wherein the first period of time has a duration of 10 to 240 seconds.

63. Method of releasing nicotine according to claim 60, wherein the second period of time has a duration of at least 60 seconds.

64. Method of releasing nicotine according to claim 60, wherein the second period of time has a duration of 5 to 30 minutes.

65. Method of releasing nicotine according to claim 60, wherein at least a part of the nicotine contained in the second module is mixed into the compressed granules comprising gum base prior to granulation.

66. Method of releasing nicotine according to claim 60, wherein at least a part of the nicotine contained in the second module is contained in compressed granules having no gum base.

67. Method of releasing nicotine according to claim 60, wherein the compressed chewing gum tablet comprises buffer in an amount of 0.01-50%.

68. Method of releasing nicotine according to claim 60, wherein a part of the second modules is comprised by compressed granules comprising one or more chewing gum ingredients selected from the group consisting of bulk sweeteners, flavors, dry binders, tabletting aids, anti-caking agents, emulsifiers, antioxidants, and any combination thereof.

69. Method of releasing nicotine according to claim 60, wherein said gum base comprises two or more ingredients selected from the group consisting of elastomers, elastomer plasticizers, resins, polyvinyl acetate, hydrogenated resins, polyterpene resins, fillers, hydrogenated starch hydrolysate, fats and waxes, and any combination thereof.

70. Method of releasing nicotine according to claim 60, wherein the first module comprising nicotine and tablet materials is formed of compressed granules of tablet material and granules comprising nicotine.

71. Method of releasing nicotine according to claim 60, wherein at least a part of the granules of the first module further comprise chewing gum ingredients selected from the group consisting of bulk sweeteners, flavors, dry binders, tabletting aids, anti-caking agents, emulsifiers, antioxidants, high intensity sweeteners, colors, and any combination thereof.

72. Method of releasing nicotine according to claim 71, wherein at least a part of the bulk sweetener of the first module is selected from the group of compressible sweeteners having a compressibility which is lower than the compressibility of the compressible bulk sweeteners of the second module.

73. Method of releasing nicotine according to claim 60, wherein the first module is comprised of compressed particles of nicotine and tablet material, wherein the tablet material comprises mucoadhesives and/or disintegrants.

74. Method of releasing nicotine according to claim 60, wherein the nicotine in the first module is non-buffered and wherein the nicotine in the second module is buffered.

75. Method of releasing nicotine according to claim 60, wherein the nicotine types in the first and second modules are different.

76. Method of releasing nicotine according to claim 60, wherein the nicotine contained in the first module comprises nicotine salts.

77. Method of releasing nicotine according to claim 60, wherein the nicotine contained in the second module comprises nicotine bound to an ion exchange resin.

78. Method of releasing nicotine according to claim 60, wherein the nicotine contained in the second module comprises nicotine bound to an inorganic filler.

79. Method of releasing nicotine according to claim 60, wherein the second module is encapsulated in the first module.

80. Method of releasing nicotine according to claim 60, wherein the first and second modules are compressed in one compressing step.

81. Chewing gum collection comprising at least two chewing gum tablets, the chewing gum tablets comprising two modules;
a first module comprising nicotine and tablet material,
a second module comprising gum base and nicotine,
the chewing gum collection further comprising instructions to a user of the chewing gum tablets,
the instructions directing a user of the chewing gum tablets to performs the steps of:
(a) positioning the chewing gum tablet in the mouth,
(b) dissolving the first module of the chewing gum tablet in a first period of time thereby releasing nicotine comprised in the first module, and thereafter
(c) chewing the remaining part of the chewing gum tablet in a second period of time thereby releasing nicotine comprised in the second module.

82. Chewing gum collection according to claim 81, wherein the chewing gum collection comprises the chewing gum tablets contained in a packaging comprising a blister packaging, a plastic container, a bag or a box.

83. Method of releasing nicotine according to claim 63, wherein the second period of time has a duration of at least 4 minutes.
